(19) United States
(12) Patent Application Publication
(10) Pub. No.: US 2011/0159092 A1
(43) Pub. Date: Jun. 30, 2011

(54) EXTENDED RELEASE PHARMACEUTICAL COMPOSITION COMPRISING LINEZOLID AND PROCESS FOR PREPARING THE SAME

(75) Inventors: Rajesh KSHIRSAGAR, Bangalore (IN); Sachin MUNDADE, Bangalore (IN); Ganesh SHINDE, Bangalore (IN); Pravin KAMBLE, Bangalore (IN); SM MUDDA, Bangalore (IN); Shivanand DHANURE, Bangalore (IN)

(73) Assignee: MICRO LABS LIMITED, Bangalore (IN)

(21) Appl. No.: 12/980,776

(22) Filed: Dec. 29, 2010

(30) Foreign Application Priority Data
Dec. 29, 2009 (IN) 3193/CHE/2009

(51) Int. Cl. A61K 31/5377; A61K 9/22; A61P 31/04

(52) U.S. Cl. 424/465; 514/236.8

(57) ABSTRACT

The present invention provides an extended release pharmaceutical composition suitable for once daily dosing comprising Linezolid or pharmaceutically acceptable salt, derivative, prodrug, metabolite and polymorph thereof and one or more pharmaceutically acceptable excipients and a process of preparing the same. The present invention further provides a method of treating bacterial infections in a mammal comprising administering an extended release, pharmaceutical composition suitable for once daily dosing comprising Linezolid capable of maintaining T>MIC for at least 24 hours. The present invention further provides an extended release pharmaceutical composition suitable for once daily dosing comprising Linezolid so that upon oral administration the maximum concentrations (Cₘₐₓ) of Linezolid in plasma are statistically significantly lower than the immediate release formulation given twice daily, and area under the plasma concentration-time curve (AUC) and the minimum plasma concentrations are maintained over 24 hours.

### FIELD OF THE INVENTION

The present invention relates to an extended release pharmaceutical composition suitable for once daily dosing comprising Linezolid or pharmaceutically acceptable salt, derivative, prodrug, metabolite and polymorph thereof and one or more pharmaceutically acceptable excipients and a process for preparing the same.

### BACKGROUND OF THE INVENTION

Linezolid is one of the oxazolidinone antibiotics with a chemical name (S)-N-({3-[3-fluoro-4-(morpholin-4-yl)phenyl]-2-oxo-1,3-oxazolidin-5-yl}methyl)acetamide it is orally effective and highly soluble in water.

Linezolid has clinical utility in the treatment of infections caused by aerobic Gram-positive bacteria. The in vitro spectrum of activity of Linezolid also includes certain Gram-negative bacteria and anaerobic bacteria. Linezolid inhibits bacterial protein synthesis through a mechanism of action different from that of other antibacterial agents, therefore, cross-resistance between Linezolid and other classes of antibiotics is unlikely. Linezolid binds to a site on the bacterial 23S ribosomal RNA of the 50S subunit and prevents the formation of a functional 70S initiation complex, which is an essential component of the bacterial translation process. The results of time-kill studies have shown Linezolid to be bacteriostatic against enterococci and staphylococci. For streptococci, Linezolid was found to be bactericidal for the majority of strains.

Linezolid is commercially sold as immediate release (IR) tablet under the trade name Zyvox®. These tablets for oral administration contain 400 mg or 600 mg Linezolid as film-coated compressed tablets and also contain corn starch, microcrystalline cellulose, hydroxypropylcellulose, sodium starch glycolate, magnesium stearate, hypromellose, polyethylene glycol, titanium dioxide, and carnauba wax. The drug appears to inhibit the initiation of protein synthesis at the ribosomal level in susceptible bacteria.

Linezolid belongs to antibiotics category which requires blood concentration of drug to be maintained above MIC (Minimum inhibitory concentration) level for longer period of time to have effective antibacterial activity. Linezolid is prescribed for the treatment of infections like community acquired pneumonia, including concurrent bacteremia, complicated skin and skin structure infections, nosocomial pneumonia at 10 to 14 days recommended duration of treatment, vancomycin resistant enterococcus faecium infections including concurrent bacteremia at 14 to 28 days recommended duration of treatment. This longer duration of treatment exposes the patient for significantly higher doses which is associated with some adverse effects like oral moniliasis, vaginal moniliasis, hypertension, dyspepsia, localized abdominal pain, pruritis and tongue discoloration, myelosupression (including anemia, leukopenia, pancytopenia and thrombocytopenia), peripheral neuropathy and optic neuropathy sometimes progressing loss of vision and lactic acidosis. Linezolid has MIC of 1-4 μg/ml.

U.S. Pat. No. 6,514,529 (“the '529 patent”) discloses immediate release tablets containing a high drug content of oxazolidinones such as Linezolid, wherein the oxazolidinone is compressed into a tablet using corn starch, microcrystalline cellulose, hydroxylpropyl cellulose, sodium starch glycolate and magnesium stearate as the excipients. The '529 patent further discloses method for providing blood levels of Linezolid by oral administration medically equivalent to the blood levels produced by IV administration of the Linezolid which comprises administration of immediate release tablet formulation.

U.S. Pat. No. 6,451,345 disclose taste masked microcapsule compositions for oxazolidinone or macrolide antibiotics comprise microcapsules of drug coated by coacervation of a microencapsulation polymer and a coated plasticized enteric polymer.

However, no reference till now discloses any extended release compositions of Linezolid suitable for once daily dosing.

Extended or sustained or slow release compositions offer clinically significant advantages for various therapeutically active agents by way of increasing patient compliance due to reduced frequency of administration; improve the safety and efficacy of drug substances and reduce undesirable effects in comparison to the corresponding immediate release dosage form.

When immediate-release compositions are given higher peak plasma concentrations are achieved this sometimes may be undesirable due to toxic effects. As extended release formulations releases the drug over prolonged period of time in controlled release manner the plasma concentrations may be achieved and maintained which are below toxic levels. Immediate release compositions in case of antibiotics due to the shorter half life eliminates faster from body and the plasma concentration drop down below MIC level and may not be effective; therefore for immediate release formulations the frequency of dosing required is more. In case of extended release formulations due to slow release of drug the in-vivo plasma concentrations are maintained above MIC level for longer period of time therefore reducing dosing frequency and improving efficacy which is desirable especially for antibiotics.

Linezolid belongs to antibiotics or antibacterial category which requires high dose of drug frequently (BID) to achieve required MIC level. For antibiotics, it is recognized that the time above minimum inhibitory concentration (T>MIC) is the pharmacodynamic parameter most closely related to efficacy and insufficient T>MIC may lead to bacterial resistance. A further parameter which may be of importance is the ratio of the maximum plasma concentration (Cₘₐₓ) to the MIC value, as this may be related to the potential to select for resistance by microorganism.

Linezolid is high dose (1200/day) and high solubility (3 mg/ml) active. These types of actives pose challenges to a formulator as it is difficult to formulate an extended release dosage form which can be suitable for once daily dosing for such actives because:
The dose of active will be high and hence there will be very little scope to include release controlling materials in the dosage form.
The large quantities of release controlling materials are required to achieve controlled drug release over the period of time and which can be suitable for once daily dosing.
Use of large quantities release controlling materials causes increase in the size of the dosage form which will make it difficult to swallow for patients.

Thus there exists an unmet need to provide a stable, simple, cost effective extended release composition comprising Linezolid without unwanted adverse effects and which maintains therapeutic plasma levels above MIC for a longer period of time.

### SUMMARY OF THE INVENTION

It is, therefore, an object of the present invention to provide an extended release pharmaceutical composition suitable for once daily dosing comprising Linezolid or pharmaceutically acceptable salt, derivative, prodrug, metabolite and polymorph thereof and one or more pharmaceutically acceptable excipients.

In yet another aspect of the present invention provides an extended release solid pharmaceutical composition suitable for once daily dosing such as tablet comprising Linezolid or pharmaceutically acceptable salt, derivative, prodrug, metabolite and polymorph thereof and one or more pharmaceutically acceptable excipients.

A further aspect of the present invention provides a process for the preparation of an extended release pharmaceutical composition suitable for once daily dosing comprising Linezolid or pharmaceutically acceptable salt, derivative, prodrug, metabolite and polymorph thereof and one or more pharmaceutically acceptable excipients.

### BRIEF DESCRIPTION OF THE DRAWING

FIG. 1 shows dissolution profiles of the pharmaceutical compositions according to the present invention for Example 1 to Example 19 using USP Type II (Paddle Apparatus) at 50 rpm in 900 ml/1000 ml, 0.01N HCl for first 2 hours, followed by the media with pH 6.8 Phosphate buffer.

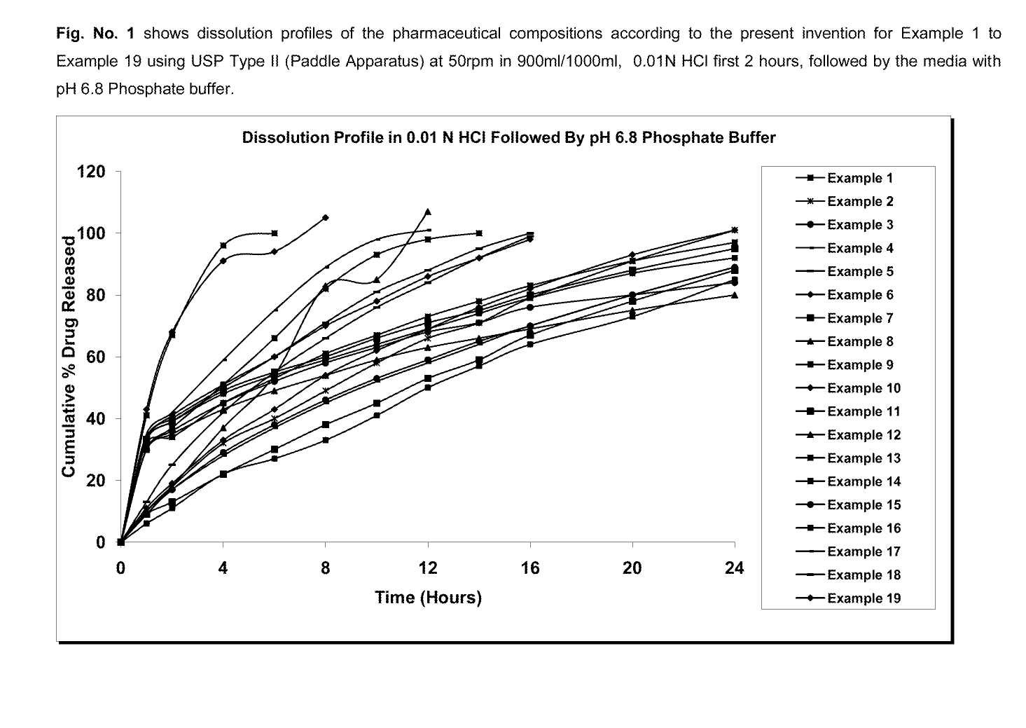

FIG. 2 shows comparative dissolution profiles of the pharmaceutical compositions according to the present invention for Example 11 and Example 15 using USP Type II (Paddle Apparatus) at 50 rpm in 900 ml/1000 ml, 0.1N HCl, pH 4.5 Acetate buffer and pH 6.8 Phosphate buffer.

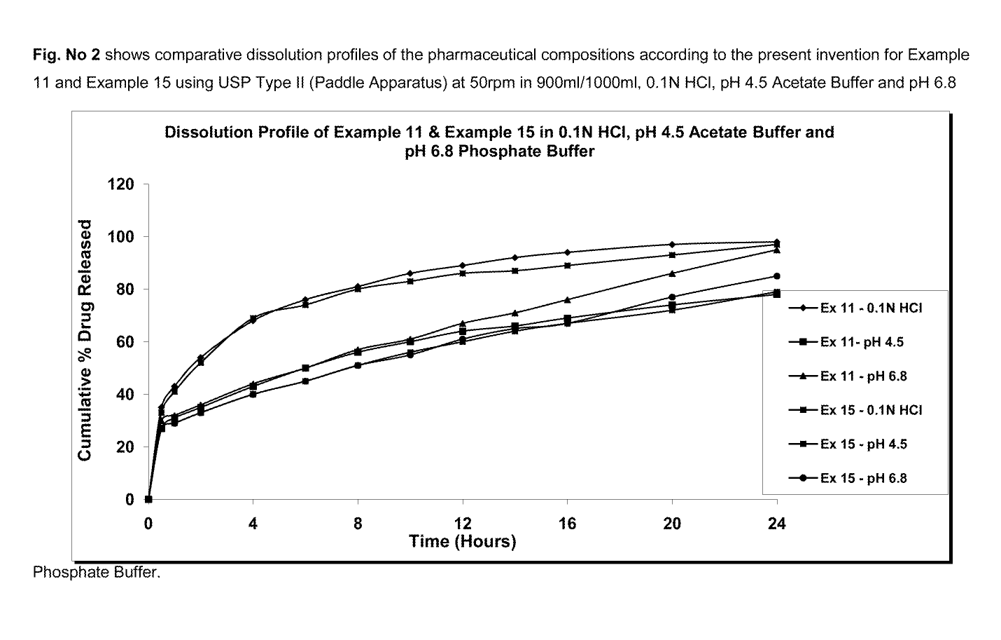

FIG. 3 shows comparative mean plasma concentration (μg/ml) vs. time (hours) profile for 1200 mg (600 mg×2 Tablets), 1000 mg ER tablets according to Example 11 (Test 1) and Example 15 (Test 2) respectively and reference immediate release Zyvox 600 mg tablet twice daily.

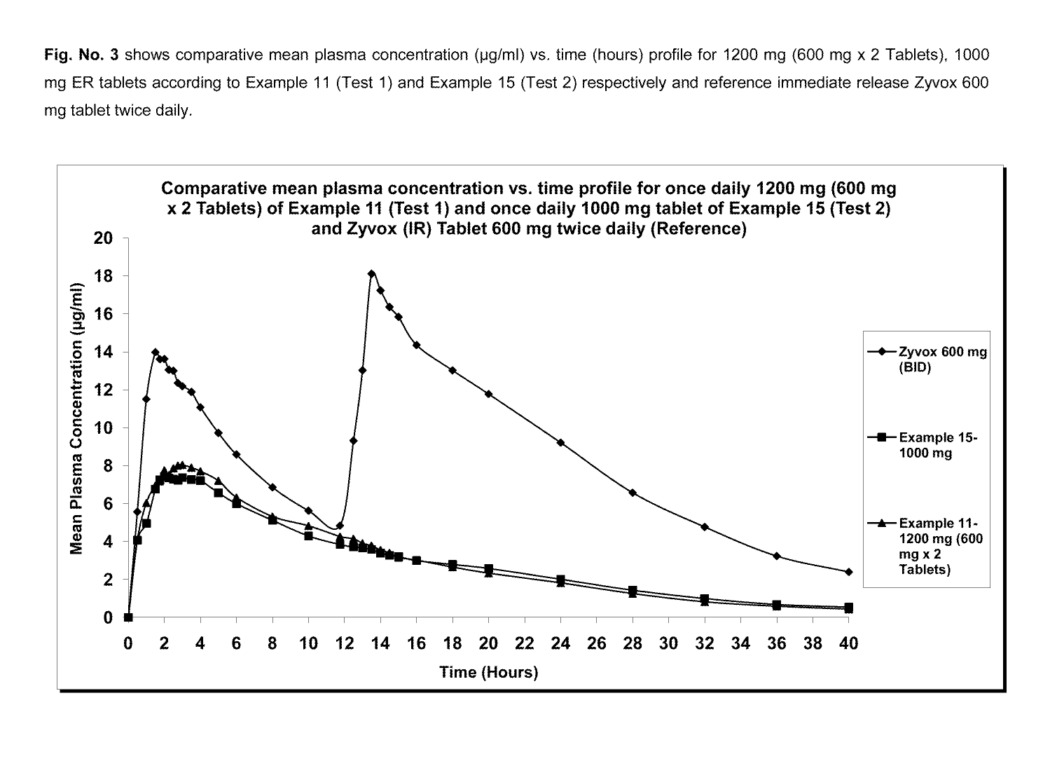

### DETAILED DESCRIPTION OF THE INVENTION

In describing and claiming the present invention, the following terminology will be used in accordance with the definitions set forth below.

The term “Linezolid” herein refers to any pharmaceutically acceptable form of Linezolid including base or its pharmaceutically acceptable complexes, salts, prodrug, derivative polymorphs, hydrates, solvates, enantiomers or racemates, metabolite as dictated by the context of its use. Linezolid may be present from about 50 mg to about 1700 mg Linezolid; preferably from about 300 mg to about 1500 mg Linezolid, preferably about 600 mg, about 1000 mg, about 1200 mg or about 1500 mg of Linezolid.

The term “extended release pharmaceutical composition” herein refers to any composition or dosage form that comprises an active drug and which is formulated to provide a longer duration of pharmacological response after administration of the dosage form than is ordinarily experienced after administration of a corresponding immediate release composition. Extended release compositions include, inter alia, those compositions described elsewhere as “controlled release”, “delayed release”, “sustained release”, “prolonged release”, “programmed release”, “time release” and/or “rate controlled” compositions or dosage forms.

The term “pharmaceutically acceptable” is meant a carrier comprised of a material that is not biologically or otherwise undesirable.

“Cₘₐₓ” as used herein, means maximum plasma concentration of Linezolid, produced by the oral administration of the composition of the invention or the immediate release (IR) comparator.

“Tₘₐₓ” as used herein, means time to the maximum observed plasma concentration.

“AUC₀₋₂₄” as used herein, means area under the plasma concentration-time curve, as calculated by the trapezoidal rule over the complete 24-hour interval for all the formulations.

“T>MIC” as used herein, means time above MIC (minimum inhibitory concentration) calculated manually by graphical interpolation, where the minimum inhibitory plasma concentration was defined as 2 μg/ml of Linezolid.

“Adverse effects” as used herein, means those physiological effects to various systems in the body such as cardiovascular systems, nervous system, digestive system, and body as a whole, which cause pain and discomfort to the individual subject.

The various embodiments of the present invention can be assembled in several different ways.

In one embodiment the present invention provides an extended release pharmaceutical composition suitable for once daily dosing comprising Linezolid or pharmaceutically acceptable salt, derivative, prodrug, metabolite and polymorph thereof and one or more pharmaceutically acceptable excipients.

In another embodiment the present invention provides an extended release tablet suitable for once daily dosing comprising Linezolid or pharmaceutically acceptable salt, derivative, prodrug, metabolite and polymorph thereof and one or more pharmaceutically acceptable excipients.

In yet another embodiment the present invention provides an extended release matrix tablet suitable for once daily dosing comprising Linezolid or pharmaceutically acceptable salt, derivative, prodrug, metabolite and polymorph thereof and one or more pharmaceutically acceptable excipients.

In yet another embodiment the present invention provides an extended release coated tablet suitable for once daily dosing comprising Linezolid or pharmaceutically acceptable salt, derivative, prodrug, metabolite and polymorph thereof and one or more pharmaceutically acceptable excipients.

In yet another embodiment the present invention provides a process of preparing an extended release pharmaceutical composition suitable for once daily dosing comprising Linezolid or pharmaceutically acceptable salt, derivative, prodrug, metabolite and polymorph thereof and one or more pharmaceutically acceptable excipients.

In yet another embodiment the present invention provides an extended release pharmaceutical composition suitable for once daily dosing comprising Linezolid or pharmaceutically acceptable salt, derivative, prodrug, metabolite and polymorph thereof and one or more release controlling material(s).

In yet another embodiment the present invention provides an extended release pharmaceutical composition suitable for once daily dosing comprising Linezolid or pharmaceutically acceptable salt, derivative, prodrug, metabolite and polymorph thereof and one or more hydrophilic release controlling material(s).

In yet another embodiment the present invention provides an extended release pharmaceutical composition suitable for once daily dosing comprising Linezolid or pharmaceutically acceptable salt, derivative, prodrug, metabolite and polymorph thereof and one or more hydrophobic release controlling material(s).

In yet another embodiment the present invention provides an extended release pharmaceutical composition suitable for once daily dosing comprising Linezolid or pharmaceutically acceptable salt, derivative, prodrug, metabolite and polymorph thereof and a matrix comprising one or more hydrophilic release controlling material(s) optionally coated with hydrophilic material and/or hydrophobic material or combinations thereof.

In yet another embodiment the present invention provides an extended release tablet suitable for once daily dosing comprising Linezolid or pharmaceutically acceptable salt, derivative, prodrug, metabolite and polymorph thereof and one or more hydrophilic release controlling material(s) optionally coated with hydrophilic material and/or hydrophobic material or combinations thereof.

In yet another embodiment the present invention provides an extended release pharmaceutical composition suitable for once daily dosing comprising Linezolid or pharmaceutically acceptable salt, derivative, prodrug, metabolite and polymorph thereof and pharmaceutically acceptable excipient having a in-vitro dissolution rate when measured using the USP Type II (Paddle apparatus) at 50 rpm in 900 ml/1000 ml, 0.01N hydrochloric acid for first 2 hours followed by the media with pH 6.8 phosphate buffer at 37° C. from about 5 to about 50% Linezolid released after 1 hour; from about 10 to about 95% Linezolid released after 4 hours; from about 35 to about 100% Linezolid released after 8 hours; from about 55 to about 100% Linezolid released after 12 hours; from about 70 to about 100% Linezolid released after 16 hours; and greater than 90% Linezolid released after 24 hours.

In yet another embodiment the present invention provides an extended release tablet comprising Linezolid having two layers comprising immediate release layer and controlled release layer; wherein immediate release layer comprises of loading dose and controlled release layer comprises of maintenance dose wherein loading dose is released immediately and maintenance dose released in controlled manner over a period of time.

In yet another embodiment the present invention provides an extended release tablet suitable for once daily dosing comprising:
a) 50-1500 mg of Linezolid or pharmaceutically acceptable salt, derivative, prodrug, and metabolite thereof;
b) 0.1% to 20% w/w of one or more release controlling materials;
c) one or more other pharmaceutically acceptable excipients.

In yet another embodiment the present invention provides an extended release pharmaceutical composition suitable for once daily dosing comprising Linezolid or pharmaceutically acceptable salt, derivative, prodrug, metabolite and polymorph thereof and one or more pharmaceutically acceptable excipients so that upon oral administration the maximum concentrations (Cₘₐₓ) of Linezolid in plasma are statistically significantly lower than the immediate release formulation given twice daily, and area under the plasma concentration-time curve (AUC) and the minimum plasma concentration are maintained over 24 hours.

In yet another embodiment the present invention provides an extended release pharmaceutical composition suitable for once daily dosing comprising Linezolid or pharmaceutically acceptable salt, derivative, prodrug, metabolite and polymorph thereof and one or more pharmaceutically acceptable excipients so that upon oral administration, the composition induces lower fluctuation in the mean plasma concentration than an immediate release composition of the Linezolid.

In yet another embodiment the present invention provides a method of using an extended release, pharmaceutical composition suitable for once daily dosing comprising Linezolid or pharmaceutically acceptable salt, derivative, prodrug, metabolite and polymorph thereof as an active ingredient, and one or more pharmaceutically acceptable excipients, comprising administering the composition in an effective amount for the treatment for bacterial infection in a mammal.

In yet another embodiment the present invention provides a method of treating bacterial infections in a mammal comprising administering an extended release, pharmaceutical composition suitable for once daily dosing comprising Linezolid or pharmaceutically acceptable salt, derivative, prodrug, metabolite and polymorph thereof as an active ingredient, and one or more pharmaceutically acceptable excipients, capable of maintaining a serum level of the Linezolid above MIC level (2 μg/ml) for at least 24 hours.

In yet another embodiment the present invention provides method of treating bacterial infections in a mammal comprising administering an extended release, pharmaceutical composition suitable for once daily dosing comprising Linezolid or pharmaceutically acceptable salt, derivative, prodrug, metabolite and polymorph thereof as an active ingredient, and one or more pharmaceutically acceptable excipients, capable of maintaining T>MIC for at least 24 hours.

In a preferred embodiment an extended release pharmaceutical composition according to present invention comprises of Linezolid from about 10% w/w to about 95% w/w of the composition, preferably from about 50% w/w to about 95% w/w of the composition.

In a preferred embodiment an extended release pharmaceutical compositions of present invention upon oral administration to a human, provides a mean maximum plasma concentration (Cₘₐₓ) of Linezolid not more than, about 16 μg/ml.

The active ingredient in the preparation according to the invention may suitably be incorporated in a matrix. This may be any matrix that affords extended release of Linezolid that affords in-vitro dissolution rates over 24 hours. Preferably the matrix is an extended release matrix. Alternatively, immediate release matrices having a release controlling coating or controlled release matrices having immediate release coating, which provides extended release of the active ingredient, may be used.

Tableting is preferred production method because it is faster, easier, adds fewer steps to the process and is the most economical. Further, the tableting method ensures a high production yield, contrary to the manufacture of pellets where the loss of production output is usually much higher. Excipients for the formulation were chosen carefully to give appropriate dissolution rate and stability of the finished dosage form.

In yet another embodiment the present invention provides, a process for preparing an extended release pharmaceutical composition comprising Linezolid or pharmaceutically acceptable salt, derivative, prodrug, and metabolite thereof and water soluble binder wherein process can be selected from direct compression, dry granulation, wet granulation (aqueous/non-aqueous or combination) and melt granulation.

In a preferred embodiment an extended release composition according to present invention comprises of one or more release controlling materials. The release controlling materials can be hydrophilic release controlling materials or hydrophobic release controlling materials. The release controlling materials are present from about 0.1% to about 20% w/w of the composition.

Examples of suitable hydrophilic release controlling materials according to present invention include but are not limited to hydroxypropyl methylcellulose, hydroxypropyl cellulose, methylcellulose, povidone, polyethylene glycols, vinyl acetate copolymers, polysaccharides such as alginates, xanthan gum, chitosan, carrageenan, dextran and the like, polyalkylene oxides such as polyethylene oxide and the likes, methacrylic acid copolymers, maleic anhydride/methyl vinyl ether copolymers, carbomers and the like.

Examples of hydrophobic release controlling materials according to present invention include but are not limited to polyvinyl acetate dispersion, ethyl cellulose, cellulose acetate, cellulose propionate (lower, medium or higher molecular weight), cellulose acetate propionate, cellulose acetate butyrate, cellulose acetate phthalate, cellulose triacetate, poly (methyl methacrylate), poly (ethyl methacrylate), poly (butyl methacrylate), poly (isobutyl methacrylate), and poly (hexyl methacrylate), poly (isodecyl methacrylate), poly (lauryl methacrylate), poly (phenyl methacrylate), poly (methyl acrylate), poly (isopropyl acrylate), poly (isobutyl acrylate), poly (octadecyl acrylate), waxes such as beeswax, carnauba wax, paraffin wax, microcrystalline wax, ozokerite; fatty alcohols such as cetostearyl alcohol, stearyl alcohol, cetyl alcohol, myristyl alcohol, and fatty acid esters such as glyceryl monostearate, glycerol monooleate, acetylated monoglycerides, tristearin, tripalmitin, cetyl esters wax, glyceryl palmitostearate, glyceryl behenate, zein and hydrogenated vegetable oils or their mixtures thereof.

The formulation will, in general comprise of one or more excipients. Examples of pharmaceutically acceptable excipients include, but are not limited to, diluents, disintegrants, lubricant, glidant, binders, fillers, surfactant, solubilizers, wetting agents, chelating agents, stabilizers, alkalizing agents or amino acids. A combination of excipients may also be used. The amount of excipient(s) employed will depend upon how much active agent is to be used. One excipient can perform more than one function.

Binders include, but are not limited to, starches such as potato starch, wheat starch, corn starch; microcrystalline cellulose such as products known under the registered trade marks Avicel, Filtrak, Heweten or Pharmacel; celluloses such as hydroxypropyl cellulose, hydroxyethyl cellulose, hydroxypropylmethyl cellulose (HPMC), ethyl cellulose, sodium carboxy methyl cellulose; natural gums like acacia, alginic acid, guar gum; liquid glucose, dextrin, povidone, syrup, polyethylene oxide, polyvinyl pyrrolidone, poly-N-vinyl amide, polyethylene glycol, gelatin, poly propylene glycol, tragacanth, combinations there of and other materials known to one of ordinary skill in the art.

Fillers or diluents, which include, but are not limited to confectioner's sugar, compressible sugar, dextrates, dextrin, dextrose, fructose, lactitol, mannitol, sucrose, starch, lactose, xylitol, sorbitol, talc, microcrystalline cellulose, calcium carbonate, calcium phosphate dibasic or tribasic, calcium sulphate, and the like can be used.

Lubricants may be selected from, but are not limited to, those conventionally known in the art such as Mg, Al, Ca or Zn stearate, polyethylene glycol, glyceryl behenate, mineral oil, sodium stearyl fumarate, stearic acid, hydrogenated vegetable oil and talc.

Glidants include, but are not limited to, silicon dioxide, magnesium trisilicate, powdered cellulose, starch, talc and tribasic calcium phosphate, calcium silicate, magnesium silicate, colloidal silicon dioxide, silicon hydrogel and other materials known to one of ordinary skill in the art.

The pharmaceutical composition according to the present invention include but is not limited to tablets (single layered tablets, multilayered tablets, mini tablets, bioadhesive tablets, caplets, matrix tablets, tablet within a tablet, mucoadhesive tablets, modified release tablets, pulsatile release tablets, timed release tablets), pellets, beads, granules, sustained release formulations, capsules, microcapsules, tablets in capsules and microspheres, matrix formulations, microencapsulation and powder/pellets/granules for suspension.

The pharmaceutical composition of the invention can optionally have one or more coatings such as film coating, sugar coating, enteric coating, bioadhesive coating and other coatings known in the art. These coatings help pharmaceutical formulations to release the drug at the required site of action.

In one example, the additional coating prevents the dosage form from contacting the mouth or esophagus. In another example, the additional coating remains intact until reaching the small intestine (e.g., an enteric coating). Premature exposure of a bioadhesive layer or dissolution of a pharmaceutical dosage form in the mouth can be prevented with a layer or coating of hydrophilic polymers such as HPMC or gelatin. Optionally, Eudragit FS 30D or other suitable polymer may be incorporated in coating composition to retard the release of the drug to ensure drug release in the colon.

These coating layers comprises one or more excipients selected from the group comprising coating agents, opacifiers, taste-masking agents, fillers, polishing agents, colouring agents, antitacking agents and the like.

Coating agents which are useful in the coating process, include, but are not limited to, polysaccharides such as maltodextrin, alkyl celluloses such as methyl or ethyl cellulose, hydroxyalkylcelluloses (e.g. hydroxypropylcellulose or hydroxypropylmethylcelluloses); polyvinylpyrrolidone, acacia, corn, sucrose, gelatin, shellac, cellulose acetate pthalate, lipids, synthetic resins, acrylic polymers, opadry, polyvinyl alcohol (PVA), copolymers of vinylpyrrolidone and vinyl acetate (e.g. marketed under the brand name of Plasdone) and polymers based on methacrylic acid such as those marketed under the brand name of Eudragit. These may be applied from aqueous or non-aqueous systems or combinations of aqueous and non-aqueous systems as appropriate. Additives can be included along with the film formers to obtain satisfactory films. These additives can include plasticizers such as dibutyl phthalate, triethyl citrate, polyethylene glycol (PEG) and the like, antitacking agents such as talc, stearic acid, magnesium stearate and colloidal silicon dioxide and the like, surfactants such as polysorbates and sodium lauryl sulphate, fillers such as talc, precipitated calcium carbonate, polishing agents such as beeswax, carnauba wax, synthetic chlorinated wax and opacifying agents such as titanium dioxide and the like. All these excipients can be used at levels well known to the persons skilled in the art.

Pharmaceutical composition of the invention can be coated by a wide variety of methods. Suitable methods include compression coating, coating in a fluidized bed or a pan and hot melt (extrusion) coating. Such methods are well known to those skilled in the art.

Non-permeable coatings of insoluble polymers, e.g., cellulose acetate, ethylcellulose, can be used as enteric coatings for delayed/modified release (DR/MR) by inclusion of soluble pore formers in the coating, e.g., PEG, PVA, sugars, salts, detergents, triethyl citrate, triacetin, etc.

Multi-layer or gradient tablets can be assembled in several different ways.

The invention will be further illustrated by the following examples, which are intended to be illustrative but not limiting.

### Example No. 1

| Ingredients | Weight (mg) |
|---|---|
| Linezolid | 600.0 |
| Microcrystalline Cellulose | 176.2 |
| Polyacrylate dispersion | 20.0 |
| 30 percent | |
| (Eudragit NE 30D) | |
| Purified water | q.s. |
| Magnesium Stearate | 3.8 |
| Total | 800.0 |

### Manufacturing Procedure:

Linezolid and microcrystalline cellulose were sifted through suitable sieve and mixed. The blend was granulated with Eudragit NE 30D and sufficient quantity of purified water. The granules were dried and sifted through suitable sieve. The Magnesium Stearate was sifted through suitable sieve and mixed with granules. The blend was compressed into tablets.

### Example No. 2

| Ingredients | Weight (mg) |
|---|---|
| Linezolid | 600.0 |
| Lactose Monohydrate | 180.2 |
| Povidone | 16.0 |
| Purified water | q.s. |
| Magnesium Stearate | 3.8 |
| Total | 800.0 |

### Manufacturing Procedure:

Linezolid, lactose monohydrate and povidone were sifted through suitable sieve and mixed. The blend was granulated with sufficient quantity of purified water. The granules were dried and sifted through suitable sieve. The Magnesium Stearate was sifted through suitable sieve and mixed with granules. The blend was compressed into tablets.

### Example No. 3

| Ingredients | Weight (mg) |
|---|---|
| Linezolid | 1200.0 |
| Lactose Monohydrate | 356.4 |
| Povidone | 16.0 |
| Purified water | q.s. |
| Magnesium Stearate | 7.6 |
| Total | 1580.0 |

### Manufacturing Procedure:

Linezolid, lactose monohydrate and povidone were sifted through suitable sieve and mixed. The blend was granulated with sufficient quantity of purified water. The granules were dried and sifted through suitable sieve. The Magnesium Stearate was sifted through suitable sieve and mixed with granules. The blend was compressed into tablets.

### Example No. 4

| Ingredients | Weight (mg) |
|---|---|
| Linezolid | 1000.0 |
| Lactose Monohydrate | 297.0 |
| Povidone | 13.3 |
| Purified water | q.s. |
| Magnesium Stearate | 6.3 |
| Total | 1316.6 |

### Manufacturing Procedure:

Linezolid, lactose monohydrate and povidone were sifted through suitable sieve and mixed. The blend was granulated with sufficient quantity of purified water. The granules were dried and sifted through suitable sieve. The Magnesium Stearate was sifted through suitable sieve and mixed with granules. The blend was compressed into tablets.

### Example No. 5

| Ingredients | Weight (mg) |
|---|---|
| Linezolid | 600.0 |
| Lactose Monohydrate | 290.0 |
| Povidone | 8.0 |
| Purified water | q.s. |
| Magnesium Stearate | 2.0 |
| Total | 900.0 |

### Manufacturing Procedure:

Linezolid, lactose monohydrate and povidone were sifted through suitable sieve and mixed. The blend was granulated with sufficient quantity of purified water. The granules were dried and sifted through suitable sieve. The Magnesium Stearate was sifted through suitable sieve and mixed with granules. The blend was compressed into tablets.

### Example No. 6

| Ingredients | Weight (mg) |
|---|---|
| Linezolid | 600.0 |
| Lactose Monohydrate | 25.0 |
| Polyethylene Glycol 6000 | 50.0 |
| Povidone | 20.0 |
| Purified water | q.s. |
| Magnesium Stearate | 5.0 |
| Total | 700.0 |

### Manufacturing Procedure:

Linezolid, Polyethylene Glycol 6000, lactose monohydrate and povidone were sifted through suitable sieve and mixed. The blend was granulated with sufficient quantity of purified water. The granules were dried and sifted through suitable sieve. The Magnesium Stearate was sifted through suitable sieve and mixed with granules. The blend was compressed into tablets.

### Example No. 7

| Ingredients | Weight (mg) |
|---|---|
| Linezolid | 600.0 |
| Lactose Monohydrate | 90.0 |
| Ethyl Cellulose | 35.0 |
| Povidone | 20.0 |
| Purified water | q.s. |
| Magnesium Stearate | 5.0 |
| Total | 750.0 |

### Manufacturing Procedure:

Linezolid, lactose monohydrate and ethylcellulose were sifted through suitable sieve and mixed. The povidone was dissolved in sufficient quantity of purified water. The blend was granulated using povidone solution. The granules were dried and sifted through suitable sieve. The Magnesium Stearate was sifted through suitable sieve and mixed with granules. The blend was compressed into tablets.

### Example No. 8

| Ingredients | Weight (mg) |
|---|---|
| Linezolid | 600.0 |
| Lactose Monohydrate | 250.0 |
| Hydroxypropyl Cellulose | 20.0 |
| Purified water | q.s. |
| Talc | 15.0 |
| Magnesium Stearate | 15.0 |
| Total | 900.0 |

### Manufacturing Procedure:

Linezolid, lactose monohydrate and hydroxypropylcellulose were sifted and mixed. The blend was granulated using sufficient quantity of purified water. The granules were dried and sifted. Talc and magnesium stearate were sifted and mixed with granules. The blend was compressed into tablets.

### Example No. 9

### Bi-Layer Composition

| | |
|---|---|
| Ingredients | Weight (mg) |

Immediate Release Layer

| | |
|---|---|
| Linezolid | 150.0 |
| Corn Starch | 15.0 |
| Microcrystalline Cellulose | 6.0 |
| Hydroxypropyl cellulose | 3.0 |
| Purified Water | q.s. |
| Microcrystalline Cellulose | 35.9 |
| Sodium Starch Glycolate | 10.5 |
| Talc | 2.35 |
| Magnesium Stearate | 2.25 |
| Total | 225.0 |

Controlled Release Layer

| | |
|---|---|
| Linezolid | 450.0 |
| Lactose monohydrate | 180.0 |
| Povidone | 22.5 |
| Purified Water | q.s. |
| Talc | 11.25 |
| Magnesium Stearate | 11.25 |
| Total | 675.0 |
| Total Weight of Bilayer | 900.0 |
| Tablet | |

### Manufacturing Procedure:

### Immediate Release Layer

Linezolid, corn starch, microcrystalline cellulose and hydroxypropylcellulose were sifted and mixed. The blend was granulated using sufficient quantity of purified water. The granules were dried, sifted and mixed with sifted microcrystalline cellulose, sodium starch glycolate. The blend was mixed with sifted talc and magnesium stearate.

### Controlled Release Layer

Linezolid, lactose monohydrate and povidone were sifted and mixed. The blend was granulated using sufficient quantity of purified water. The granules were dried and sifted. The magnesium stearate was sifted and mixed with granules.

The blend of immediate release layer was compressed over the pre-compressed blend of controlled release layer to form a bilayer tablets.

### Example No. 10

| Ingredients | Weight (mg) |
|---|---|
| Linezolid | 600.0 |
| Lactose Monohydrate | 260.0 |
| Hydroxypropyl Cellulose | 20.0 |
| Purified Water | q.s. |
| Talc | 10.0 |
| Magnesium Stearate | 10.0 |
| Total | 900.0 |

### Manufacturing Procedure:

Linezolid, lactose monohydrate and hydroxypropylcellulose were sifted and mixed. The blend was granulated using sufficient quantity of purified water. The granules were dried and sifted. Talc and magnesium stearate was sifted and mixed with granules. The blend was compressed into tablets.

### Example No. 11

### Bi-Layer Composition

| Ingredients | Weight (mg) |
|---|---|
| Strength | 600 mg |

Immediate Release Layer

| | |
|---|---|
| Linezolid | 150.0 |
| Microcrystalline Cellulose | 6.0 |
| Hypromellose | 3.0 |
| Carboxymethylcellulose | 15.0 |
| Calcium | |
| Purified Water | q.s. |
| Microcrystalline Cellulose | 36.0 |
| Sodium Starch Glycolate | 10.5 |
| Talc | 2.25 |
| Magnesium Stearate | 2.25 |
| Total | 225.0 |

Controlled Release Layer

| | |
|---|---|
| Linezolid | 450.0 |
| Lactose monohydrate | 180.0 |
| Povidone | 22.5 |
| Purified Water | q.s. |
| Talc | 11.25 |
| Magnesium Stearate | 11.25 |
| Total | 675.0 |
| Total Weight of Bilayer | 900.0 |
| Tablet | |

### Manufacturing Procedure:

### Immediate Release Layer

Linezolid, microcrystalline cellulose, hypromellose and Carboxymethylcellulose Calcium were sifted and mixed. The blend was granulated using sufficient quantity of purified water. The granules were dried, sifted and mixed with sifted microcrystalline cellulose, sodium starch glycolate. The blend was mixed with sifted talc and magnesium stearate.

### Controlled Release Layer

Linezolid, lactose monohydrate and povidone were sifted and mixed. The blend was granulated using sufficient quantity of purified water. The granules were dried and sifted. The granules were mixed with sifted talc and magnesium stearate.

The blend of immediate release layer was compressed over the pre-compressed blend of controlled release layer to form a bilayer tablet.

### Example No. 12

### Bi-Layer Composition

| Ingredients | Weight (mg) |
|---|---|
| Strength | 1000 mg |

Immediate Release Layer

| | |
|---|---|
| Linezolid | 250.0 |
| Microcrystalline Cellulose | 10.0 |
| Hypromellose | 5.0 |
| Carboxymethylcellulose | 25.0 |
| Calcium | |
| Purified Water | q.s. |
| Microcrystalline Cellulose | 60.0 |
| Sodium Starch Glycolate | 17.5 |
| Talc | 3.75 |
| Magnesium Stearate | 3.75 |
| Total | 375.0 |

Controlled Release Layer

| | |
|---|---|
| Linezolid | 750.0 |
| Lactose monohydrate | 300.0 |
| Povidone | 37.5 |
| Purified Water | q.s. |
| Talc | 18.75 |
| Magnesium Stearate | 18.75 |
| Total | 1125.0 |
| Total Weight of Bilayer | 1500.0 |
| Tablet | |

### Manufacturing Procedure:

### Immediate Release Layer

Linezolid, microcrystalline cellulose, hypromellose and Carboxymethylcellulose Calcium were sifted and mixed. The blend was granulated using sufficient quantity of purified water. The granules were dried, sifted and mixed with sifted microcrystalline cellulose, sodium starch glycolate. The blend was mixed with sifted talc and magnesium stearate.

### Controlled Release Layer

Linezolid, lactose monohydrate and povidone were sifted and mixed. The blend was granulated using sufficient quantity of purified water. The granules were dried and sifted. The granules were mixed with sifted talc and magnesium stearate.

The blend of immediate release layer was compressed over the pre-compressed blend of controlled release layer to form a bilayer tablet.

### Example No. 13

### Compression Coated Tablet (Tablet in Tablet) Composition

| | |
|---|---|
| Ingredients | Weight (mg) |

Controlled Release Inner Core

| | |
|---|---|
| Linezolid | 450.0 |
| Lactose monohydrate | 73.0 |
| Povidone | 15.0 |
| Purified Water | q.s. |
| Talc | 6.0 |
| Magnesium Stearate | 6.0 |
| Total Controlled Release Core | 550.0 |

Immediate Release outer compression Coat

| | |
|---|---|
| Linezolid | 150.0 |
| Microcrystalline Cellulose | 500.0 |
| Carboxymethylcellulose | 60.0 |
| Calcium | |
| Hypromellose | 15.0 |
| Purified Water | q.s. |
| Microcrystalline Cellulose | 210.0 |
| Sodium Starch Glycolate | 45.0 |
| Talc | 10.0 |
| Magnesium Stearate | 10.0 |
| Total | 1000.0 |
| Total weight of compression | 1550.0 |
| coated tablet | |

### Manufacturing Procedure:

### Controlled Release Inner Core

Linezolid, lactose monohydrate and povidone were sifted and mixed. The blend was granulated using sufficient quantity of purified water. The granules were dried and sifted. The granules were mixed with sifted talc and magnesium stearate. The blend was compressed into tablet.

### Immediate Release Outer Compression Coat

Linezolid, microcrystalline cellulose, hypromellose and Carboxymethylcellulose Calcium were sifted and mixed. The blend was granulated using sufficient quantity of purified water. The granules was dried, sifted and mixed with sifted microcrystalline cellulose, sodium starch glycolate. The blend was mixed with sifted talc and magnesium stearate. The inner core tablets were compression coated with the immediate release blend to form a controlled release tablet inside the immediate release tablets.

### Example No. 14

### Bi-Layer Composition

| Ingredients | Weight (mg) |
|---|---|
| Strength | 1000 mg |

Immediate Release Layer

| | |
|---|---|
| Linezolid | 250.0 |
| Microcrystalline Cellulose | 10.0 |
| Hypromellose | 5.0 |
| Carboxymethylcellulose | 25.0 |
| Calcium | |
| Purified Water | q.s. |
| Microcrystalline Cellulose | 60.0 |
| Sodium Starch Glycolate | 17.5 |
| Talc | 3.75 |
| Magnesium Stearate | 3.75 |
| Total | 375.0 |

Controlled Release Layer

| | |
|---|---|
| Linezolid | 750.0 |
| Lactose monohydrate | 312.5 |
| Hydroxypropyl Cellulose | 25.0 |
| Purified Water | q.s. |
| Talc | 18.75 |
| Magnesium Stearate | 18.75 |
| Total | 1125.0 |
| Total Weight of Bilayer | 1500.0 |
| Tablet | |

### Manufacturing Procedure:

### Immediate Release Layer

Linezolid, microcrystalline cellulose, hypromellose and Carboxymethylcellulose Calcium were sifted and mixed. The blend was granulated using sufficient quantity of purified water. The granules were dried, sifted and mixed with sifted microcrystalline cellulose, sodium starch glycolate. The blend was mixed with sifted talc and magnesium stearate.

### Controlled Release Layer

Linezolid, lactose monohydrate and hydroxypropylcellulose were sifted and mixed. The blend was granulated using sufficient quantity of purified water. The granules were dried and sifted. The granules were mixed with sifted talc and magnesium stearate.

The blend of immediate release layer was compressed over the pre-compressed blend of controlled release layer to form a bilayer tablet.

### Example No. 15

### Bi-Layer Composition

| Ingredients | Weight (mg) |
|---|---|
| Strength | 1000 mg |

Immediate Release Layer

| | |
|---|---|
| Linezolid | 250.0 |
| Microcrystalline Cellulose | 10.0 |
| Hypromellose | 5.0 |
| Carboxymethylcellulose | 25.0 |
| Calcium | |
| Purified Water | q.s. |
| Microcrystalline Cellulose | 60.0 |
| Sodium Starch Glycolate | 17.5 |
| Talc | 3.75 |
| Magnesium Stearate | 3.75 |
| Total | 375.0 |

Controlled Release Layer

| | |
|---|---|
| Linezolid | 750.0 |
| Lactose monohydrate | 105.0 |
| Povidone | 25.0 |
| Purified Water | q.s. |
| Talc | 10.0 |
| Magnesium Stearate | 10.0 |
| Total | 900.0 |
| Total Weight of Bilayer | 1275.0 |
| Tablet | |

### Manufacturing Procedure:

### Immediate Release Layer

Linezolid, microcrystalline cellulose, hypromellose and Carboxymethylcellulose Calcium were sifted and mixed. The blend was granulated using sufficient quantity of purified water. The granules were dried, sifted and mixed with sifted microcrystalline cellulose, sodium starch glycolate. The blend was mixed with sifted talc and magnesium stearate.

### Controlled Release Layer

Linezolid, lactose monohydrate and povidone were sifted and mixed. The blend was granulated using sufficient quantity of purified water. The granules were dried and sifted. The granules were mixed with sifted talc and magnesium stearate.

The blend of immediate release layer was compressed over the pre-compressed blend of controlled release layer to form a bilayer tablet.

### Example No. 16

| | |
|---|---|
| Ingredients | Weight (mg) |

Core Tablet

| | |
|---|---|
| Linezolid | 600.0 |
| Microcrystalline Cellulose | 190.0 |
| HPMC | 200.0 |
| Purified Water | q.s. |
| Talc | 10.0 |
| Magnesium Stearate | 10.0 |
| Total | 1010.0 |

### Manufacturing Procedure:

Linezolid, microcrystalline cellulose and HPMC were sifted and mixed. The blend was granulated with purified water. The granules were dried, sifted and mixed with sifted talc and magnesium stearate. The blend was compressed into tablets.

### Example 17

### Bilayer Tablet

| Ingredients | Weight (mg) |
|---|---|
| Strength | 600 mg |

Immediate Release Layer

| | |
|---|---|
| Linezolid | 150.0 |
| Dibasic Calcium Phosphate | 48.0 |
| Croscarmellose Sodium | 5.0 |
| Carboxymethylcellulose | 15.0 |
| Calcium | |
| Purified Water | q.s. |
| Croscarmellose Sodium | 5.0 |
| Magnesium Stearate | 2.0 |
| Total | 225.0 |

Controlled Release Layer

| | |
|---|---|
| Linezolid | 450.0 |
| Lactose monohydrate | 180.0 |
| Hydroxypropylcellulose | 22.5 |
| Purified Water | q.s. |
| Talc | 11.25 |
| Magnesium Stearate | 11.25 |
| Total | 675.0 |
| Total Weight of Bilayer | 900.0 |
| Tablet | |

Manufacturing Procedure:

### Immediate Release Layer

Linezolid, dibasic calcium phosphate, croscarmellose sodium and carboxymethylcellulose calcium were sifted and mixed. The blend was granulated using sufficient quantity of purified water. The granules were dried, sifted and mixed with sifted croscarmellose sodium. The blend was mixed with sifted magnesium stearate.

### Controlled Release Layer

Linezolid, lactose monohydrate and hydroxypropylcellulose were sifted and mixed. The blend was granulated using sufficient quantity of purified water. The granules were dried and sifted. The granules were mixed with sifted talc and magnesium stearate.

The blend of immediate release layer was compressed over the pre-compressed blend of controlled release layer to form a bilayer tablets.

### Example 18

### Bilayer Tablet

| Ingredients | Weight (mg) |
|---|---|
| Strength | 1000 mg |

Immediate Release Layer

| | |
|---|---|
| Linezolid | 250.0 |
| Dibasic Calcium Phosphate | 80.0 |
| Croscarmellose Sodium | 8.3 |
| Carboxymethylcellulose | 25.0 |
| Calcium | |
| Purified Water | q.s. |
| Croscarmellose Sodium | 8.3 |
| Magnesium Stearate | 3.4 |
| Total | 375.0 |

Controlled Release Layer

| | |
|---|---|
| Linezolid | 750.0 |
| Lactose monohydrate | 300.0 |
| Hydroxypropylcellulose | 37.5 |
| Purified Water | q.s. |
| Talc | 18.75 |
| Magnesium Stearate | 18.75 |
| Total | 1125.0 |
| Total Weight of Bilayer | 1500.0 |
| Tablet | |

### Manufacturing Procedure:

### Immediate Release Layer

Linezolid, dibasic calcium phosphate, croscarmellose sodium and carboxymethylcellulose calcium was sifted and mixed. The blend was granulated using sufficient quantity of purified water. The granules was dried, sifted and mixed with sifted croscarmellose sodium. The blend was mixed with sifted magnesium stearate.

### Controlled Release Layer

Linezolid, lactose monohydrate and hydroxypropylcellulose were sifted and mixed. The blend was granulated using sufficient quantity of purified water. The granules were dried and sifted. The granules were mixed with sifted talc and magnesium stearate.

The blend of immediate release layer was compressed over the pre-compressed blend of controlled release layer to form a bilayer tablets.

### Example 19

### Bilayer Tablet

| Ingredients | Weight (mg) |
|---|---|
| Strength | 1000 mg |

Immediate Release Layer

| | |
|---|---|
| Linezolid | 250.0 |
| Dibasic Calcium Phosphate | 80.0 |
| Croscarmellose Sodium | 8.3 |
| Carboxymethylcellulose | 25.0 |
| Calcium | |
| Purified Water | q.s. |
| Croscarmellose Sodium | 8.3 |
| Magnesium Stearate | 3.4 |
| Total | 375.0 |

Controlled Release Layer

| | |
|---|---|
| Linezolid | 750.0 |
| Lactose monohydrate | 105.0 |
| Hydroxypropylcellulose | 25.0 |
| Purified Water | q.s. |
| Talc | 10.0 |
| Magnesium Stearate | 10.0 |
| Total | 900.0 |
| Total Weight of Bilayer | 1275.0 |
| Tablet | |

### Manufacturing Procedure:

### Immediate Release Layer

Linezolid, dibasic calcium phosphate, croscarmellose sodium and carboxymethylcellulose calcium were sifted and mixed. The blend was granulated using sufficient quantity of purified water. The granules were dried, sifted and mixed with sifted croscarmellose sodium. The blend was mixed with sifted magnesium stearate.

### Controlled Release Layer

Linezolid, lactose monohydrate and hydroxypropylcellulose were sifted and mixed. The blend was granulated using sufficient quantity of purified water. The granules were dried and sifted. The granules were mixed with sifted talc and magnesium stearate.

The blend of immediate release layer was compressed over the pre-compressed blend of controlled release layer to form a bilayer tablets.

### Example No. 20

### Coated Tablet

| | |
|---|---|
| Ingredients | Weight (mg) |

Core Tablet

| | |
|---|---|
| Linezolid | 600.0 |
| Corn Starch | 60.0 |
| Microcrystalline Cellulose | 24.0 |
| Hydroxypropylcellulose | 12.0 |
| Purified Water | q.s. |
| Microcrystalline Cellulose | 153.6 |
| Sodium Starch Glycolate | 42.0 |
| Magnesium Stearate | 8.4 |
| Total | 900.0 |

Controlled Release Coating

| | |
|---|---|
| Ethylcellulose based | 45.0 |
| coating material | |
| (Surelease) | |
| HPMC based coating | 6.75 |
| material (Opadry clear) | |
| Purified Water | q.s. |
| Total coating | 51.75 |

### Manufacturing Procedure:

### Core Tablet

Linezolid, corn starch, microcrystalline cellulose and hydroxypropylcellulose were sifted and mixed. The blend was granulated with sufficient quantity of purified water. The granules were dried, sifted and mixed with microcrystalline cellulose and sodium starch glycolate. The blend was mixed with magnesium stearate and compressed into tablets.

Coating: HPMC based coating material (Opadry Clear) was dissolved in sufficient quantity of purified water and mixed with ethylcellulose based aqueous coating material (Surelease). The core tablets were coated with the coating solution to a desired weight gain.

### Example No. 21

### Coated Tablet

| | |
|---|---|
| Ingredients | Weight (mg) |

Core Tablet

| | |
|---|---|
| Linezolid | 600.0 |
| Corn Starch | 60.0 |
| Microcrystalline Cellulose | 24.0 |
| Hydroxypropylcellulose | 12.0 |
| Purified Water | q.s. |
| Microcrystalline Cellulose | 143.0 |
| Sodium Starch Glycolate | 42.0 |
| Talc | 10.0 |
| Magnesium Stearate | 9.0 |
| Total | 900.0 |

Controlled Release Coating

| | |
|---|---|
| Ethylcellulose | 45.0 |
| HPMC | 18.0 |
| Triethylcitrate | 4.5 |
| Isopropyl Alcohol | q.s. |
| Purified Water | q.s. |
| Total coating | 67.5 |

### Manufacturing Procedure:

### Core Tablet

Linezolid, corn starch, microcrystalline cellulose and hydroxypropylcellulose were sifted and mixed. The blend was granulated with sufficient quantity of purified water. The granules were dried, sifted and mixed with microcrystalline cellulose and sodium starch glycolate. The blend was mixed with magnesium stearate and compressed into tablets.

Coating: HPMC, triethylcitrate was dissolved in sufficient quantity of purified water, ethylcellulose was dissolved in isopropyl alcohol. Both the solutions were mixed and the core tablets were coated to a desired weight gain.

### Example No. 22

### Pharmacokinetic Study of the Extended Release Linezolid Formulation

The pharmacokinetic study to determine the concentration-time plasma profile was done on healthy male subjects. The study was conducted as open label, balanced, randomized, three-treatment, single-period, parallel, comparative oral bioavailability study as described below:

### Single Dose Study:

A total of 18 normal, healthy, adult, male subjects were enrolled in the study. Healthy male subjects were aged between 18 and 40 years with BMI between 19-24 Kg/m² but body weight not less than 45 Kgs.

The Linezolid ER tablets 1200 mg (600 mg×2 tablets as a single dose) and Linezolid ER tablets 1000 mg (as a single dose) and Linezolid 600 mg IR Tablets (reference product) currently sold by Pharmacia Limited under the Trade name Zyvox® (2 times at the interval of 12 hours (2 doses)) were administered to 18 healthy subjects under fasting condition in the morning.

The study was conducted according to open label, balanced, randomized, three-treatment, single-period, parallel, comparative oral bioavailability study.

The blood samples (5 ml each) were withdrawn at pre-dose (before dosing, in the morning of the day of dosing) and at 0.5, 1.0, 2.0, 3.0, 4.0, 6.0, 8.0, 12.0, 16.0, 24.0, 36.0 and 40.0 hours after morning dosing. Plasma samples were analyzed to quantify the concentrations of Linezolid using a validated LC/MS/MS bioanalytical method.

### Pharmacokinetic Analyses:

Values for Linezolid pharmacokinetic parameters including observed AUC₀₋₂₄ Cₘₐₓ, Tₘₐₓ, were calculated using standard non-compartmental methods. The time above the minimum inhibitory plasma concentration (T>MIC) was calculated manually by graphical interpolation, where the minimum inhibitory plasma concentration was defined as 2 μg/ml of Linezolid.

The in vitro dissolution is carried out using USP Type II (Paddle Apparatus) at 50 rpm in 900 ml/1000 ml, 0.01N HCl first two hours, followed by the media with pH 6.8 Phosphate buffer. The release of drug is measured using UV techniques. The dissolution pattern for Example No. 1 to Example No. 9 is given in the table below

| | Dissolution | | | | | | | | |
|---|---|---|---|---|---|---|---|---|---|
| | Dissolution Media: 900 ml/1000 ml 0.01N HCl for first 2 hours | | | | | | | | |
| | followed by media pH 6.8 Phosphate Buffer, USP II, 50 RPM | | | | | | | | |
| | Example No. | | | | | | | | |
| | 1 | 2 | 3 | 4 | 5 | 6 | 7 | 8 | 9 |
| Time (Hours) | Cumulative % Drug Released | | | | | | | | |
| 1 | 41 | 10 | 9 | 9 | 13 | 11 | 9 | 10 | 33 |
| 2 | 67 | 18 | 17 | 17 | 25 | 19 | 13 | 18 | 39 |
| 4 | 96 | 32 | 29 | 28 | 42 | 33 | 22 | 37 | 48 |
| 6 | 100 | 40 | 38 | 37 | 55 | 43 | 30 | 54 | 54 |
| 8 | — | 49 | 46 | 45 | 66 | 54 | 38 | 83 | 59 |
| 10 | — | 58 | 53 | 52 | 76 | 62 | 45 | 85 | 64 |
| 12 | — | 66 | 59 | 58 | 84 | 69 | 53 | 107 | 69 |
| 14 | — | 71 | 65 | 64 | 92 | 76 | 59 | — | 74 |
| 16 | — | 79 | 70 | 70 | 99 | 82 | 67 | — | 79 |
| 20 | — | 91 | 80 | 80 | — | 93 | 78 | — | 87 |
| 24 | — | 101 | 89 | 89 | — | 101 | 88 | — | 92 |

The in vitro dissolution is carried out using USP Type II (Paddle Apparatus) at 50 rpm in 900 ml/1000 ml, 0.01N HCl first two hours, followed by the media with pH 6.8 Phosphate buffer. The release of drug is measured using UV techniques. The dissolution pattern for Example No. 10 to Example No. 19 is given in the table below:

| | Dissolution | | | | | | | | | |
|---|---|---|---|---|---|---|---|---|---|---|
| | Dissolution Media: 900 ml/1000 ml, 0.01N HCl for first 2 hours | | | | | | | | | |
| | followed by media pH 6.8 Phosphate Buffer, USP II, 50 RPM | | | | | | | | | |
| | Example | | | | | | | | | |
| | 10 | 11 | 12 | 13 | 14 | 15 | 16 | 17 | 18 | 19 |
| Time (hours) | Cumulative % Drug Released | | | | | | | | | |
| 1 | 43 | 33 | 30 | 32 | 30 | 30 | 6 | 35 | 34 | 33 |
| 2 | 68 | 39 | 35 | 34 | 37 | 36 | 11 | 42 | 40 | 41 |
| 4 | 91 | 49 | 43 | 45 | 51 | 45 | 22 | 59 | 50 | 51 |
| 6 | 94 | 55 | 49 | 53 | 66 | 52 | 27 | 75 | 60 | 60 |
| 8 | 105 | 60 | 54 | 61 | 82 | 58 | 33 | 89 | 71 | 70 |
| 10 | — | 66 | 59 | 67 | 93 | 63 | 41 | 98 | 81 | 78 |
| 12 | — | 71 | 63 | 73 | 98 | 68 | 50 | 101 | 88 | 86 |
| 14 | — | 75 | 66 | 78 | 100 | 71 | 57 | — | 95 | 92 |
| 16 | — | 80 | 69 | 83 | — | 76 | 64 | — | 100 | 98 |
| 20 | — | 88 | 75 | 91 | — | 80 | 73 | — | — | — |
| 24 | — | 95 | 80 | 97 | — | 84 | 85 | — | — | — |

The in vitro dissolution is carried out using USP Type II (Paddle Apparatus) at 50 rpm in 900 ml/1000 ml, 0.1 N HCl, pH 4.5 Acetate Buffer and pH 6.8 Phosphate Buffer. The release of drug is measured using UV techniques. The dissolution pattern for Example No. 11 and Example No. 15 is given in the table below:

| | Strength | | | | | |
|---|---|---|---|---|---|---|
| | 600 mg | 1000 mg | 600 mg | 1000 mg | 600 mg | 1000 mg |
| | Example No. | | | | | |
| | 11 | 15 | 11 | 15 | 11 | 15 |
| Media | 0.1N HCl | | pH 4.5 Acetate Buffer | | pH 6.8 Phosphate Buffer | |
| Volume | 900 ml | 1000 ml | 900 ml | 1000 ml | 900 ml | 1000 ml |
| Condition | USP II (Paddle), 50 RPM | | | | | |
| Time (Hrs) | Cumulative % Drug Release | | | | | |
| 0.5 | 35 | 33 | 27 | 27 | 30 | 27 |
| 1 | 43 | 41 | 31 | 29 | 32 | 29 |
| 2 | 54 | 52 | 35 | 33 | 36 | 33 |
| 4 | 68 | 69 | 43 | 40 | 44 | 40 |
| 6 | 76 | 74 | 50 | 45 | 50 | 45 |
| 8 | 81 | 80 | 56 | 51 | 57 | 51 |
| 10 | 86 | 83 | 60 | 56 | 61 | 55 |
| 12 | 89 | 86 | 64 | 60 | 67 | 61 |
| 14 | 92 | 87 | 66 | 64 | 71 | 65 |
| 16 | 94 | 89 | 69 | 67 | 76 | 67 |
| 20 | 97 | 93 | 74 | 72 | 86 | 77 |
| 24 | 98 | 97 | 78 | 79 | 95 | 85 |

Values for Linezolid pharmacokinetic parameters as per pharmacokinetic study conducted in Example 22 including observed Cₘₐₓ, Tₘₐₓ, AUC₀₋₂₄ T>MIC are given in below table.

| | | Tₘₐₓ (hrs) | AUC₀₋₂₄ | |
|---|---|---|---|---|
| | Cₘₐₓ (μg/ml) | Median | (μg · h/ml) | T > MIC |
| Formulation | Mean ± SD | (Range) | Mean ± SD | (hrs) |
| Example 11 Linezolid ER Tablet 1200 mg | 8.16 ± 0.69 | 2.9(1.5-4.0) | 119.70 ± 9.58 | 24 |
| (i.e. 600 mg × 2 tablets) as single dose. | | | | |
| Example 15 Linezolid ER Tablet (1000 mg) | 7.84 ± 1.06 | 2.0(0.5-3.5) | 117.60 ± 38.00 | 24 |
| single dose. | | | | |
| Reference Zyvox ® (600 mg) 2 times at the | 18.78 ± 3.58 | 1.5(0.5-2.5) | 334.72 ± 45.61 | 24 |
| interval of 12 hours (2 doses) | | | | |

We claim:

1. An extended release pharmaceutical composition suitable for once daily dosing comprising Linezolid or pharmaceutically acceptable salt, derivative, prodrug, metabolite and polymorph thereof and one or more pharmaceutically acceptable excipients.

2. An extended release pharmaceutical composition according to claim 1 wherein the Linezolid is present from about 50 mg to about 1700 mg.

3. An extended release pharmaceutical composition according to claim 1 wherein the Linezolid is present from about 300 mg to about 1500 mg.

4. An extended release pharmaceutical composition according to claim 1 wherein the Linezolid is present from about 10% w/w to about 95% w/w of the composition.

5. An extended release pharmaceutical composition according to claim 1 wherein the Linezolid is present from about 50% w/w to about 95% w/w of the composition.

6. An extended release pharmaceutical composition according to claim 1 which upon oral administration to a human provides a mean maximum plasma concentration (Cₘₐₓ) of Linezolid not more than about 16 μg/ml.

7. An extended release pharmaceutical composition according to claim 1 which upon oral administration to a human provides a mean plasma concentration of at least 2 μg/ml of Linezolid for at least 24 hours.

8. A method of reducing adverse effects comprising administering pharmaceutical composition according to claim 1.

9. An extended release pharmaceutical composition according to claim 1 which upon oral administration induces lower fluctuation in the mean plasma concentration than an immediate release composition of the Linezolid.

10. An extended release pharmaceutical composition according to claim 1 wherein pharmaceutical composition comprises of one or more release controlling materials.

11. An extended release pharmaceutical composition according to claim 10 wherein release controlling materials are present from about 0.1% w/w to about 20% w/w of the composition.

12. An extended release pharmaceutical composition according to claim 10 wherein the release controlling material is selected from group consisting of hydrophilic release controlling material or hydrophobic release controlling material or combinations thereof.

13. An extended release pharmaceutical composition according to claim 12 wherein hydrophilic release controlling material is selected from group consisting of hydroxypropyl methylcellulose, hydroxypropyl cellulose, methylcellulose, povidone, polyethylene glycols, vinyl acetate copolymers, polysaccharides as alginates, xanthan gum, chitosan, carrageenan, dextran, polyalkylene oxides as polyethylene oxide, methaacrylic acid copolymers, maleic anhydride/methyl vinyl ether copolymers, carbomers.

14. An extended release pharmaceutical composition according to claim 12 wherein hydrophobic release controlling material is selected from group consisting of polyvinyl acetate dispersion, ethyl cellulose, cellulose acetate, cellulose propionate (lower, medium or higher molecular weight), cellulose acetate propionate, cellulose acetate butyrate, cellulose acetate phthalate, cellulose triacetate, poly (methyl methacrylate), poly (ethyl methacrylate), poly (butyl methacrylate), poly (isobutyl methacrylate), poly (hexyl methacrylate), poly (isodecyl methacrylate), poly (lauryl methacrylate), poly (phenyl methacrylate), poly (methyl acrylate), poly (isopropyl acrylate), poly (isobutyl acrylate), poly (octadecyl acrylate), beeswax, carnauba wax, paraffin wax, microcrystalline wax, ozokerite; cetostearyl alcohol, stearyl alcohol, cetyl alcohol; myristyl alcohol, glyceryl monostearate; glycerol monooleate, acetylated monoglycerides, tristearin, tripalmitin, cetyl esters wax, glyceryl palmitostearate, glyceryl behenate, zein and hydrogenated vegetable oils.

15. An extended release pharmaceutical composition according to claim 12 wherein the composition additionally contain other pharmaceutically acceptable excipients such as fillers, binders, and lubricants.

16. An extended release pharmaceutical composition suitable for once daily dosing comprising Linezolid or pharmaceutically acceptable salt, derivative, prodrug, metabolite and polymorph thereof having a in-vitro dissolution rate when measured using the USP Type II (Paddle apparatus) at 50 rpm in 900 ml/1000 ml, 0.01N hydrochloric acid for first 2 hours followed by the media with pH 6.8 phosphate buffer at 37° C.
from about 5 to about 50% Linezolid released after 1 hour;
from about 10 to about 95% Linezolid released after 4 hours;
from about 35 to about 100% Linezolid released after 8 hours;
from about 55 to about 100% Linezolid released after 12 hours;
from about 70 to about 100% Linezolid released after 16 hours;
and greater than 90% Linezolid released after 24 hours.

17. An extended release pharmaceutical composition according to claim 16 wherein the Linezolid is present from about 50 mg to about 1700 mg.

18. An extended release pharmaceutical composition according to claim 16 wherein the Linezolid is present from about 300 mg to about 1500 mg.

19. An extended release pharmaceutical composition according to claim 16 wherein the Linezolid is present from about 10% w/w to about 95% w/w of the composition.

20. An extended release pharmaceutical composition according to claim 16 wherein the Linezolid is present from about 50% w/w to about 95% w/w of the composition.

21. An extended release pharmaceutical composition according to claim 16 which, upon oral administration to a human, provides a mean maximum plasma concentration (Cₘₐₓ) of Linezolid not more than about 16 μg/ml.

22. An extended release pharmaceutical composition according to claim 16 which, upon oral administration to a human, provides a mean plasma concentration of at least 2 μg/ml of Linezolid for at least 24 hours.

23. An extended release pharmaceutical composition according to claim 16 wherein pharmaceutical composition comprises of one or more release controlling materials.

24. An extended release pharmaceutical composition according to claim 23 wherein release controlling materials are present from about 0.1% w/w to about 20% w/w of the composition.

25. An extended release pharmaceutical composition according to claim 23 wherein release controlling material is selected from group consisting of hydrophilic release controlling materials or hydrophobic release controlling materials or combinations thereof.

26. An extended release pharmaceutical composition according to claim 25 wherein hydrophilic release controlling material is selected from group consisting of hydroxypropyl methylcellulose, hydroxypropyl cellulose, methylcellulose, povidone, polyethylene glycols, vinyl acetate copolymers, polysaccharides as alginates, xanthan gum, chitosan, carrageenan, dextran, polyalkylene oxides as polyethylene oxide, methaacrylic acid copolymers, maleic anhydride/methyl vinyl ether copolymers, carbomers.

27. An extended release pharmaceutical composition according to claim 25 wherein hydrophobic material is selected from group consisting of polyvinyl acetate dispersion, ethyl cellulose, cellulose acetate, cellulose propionate (lower, medium or higher molecular weight), cellulose acetate propionate, cellulose acetate butyrate, cellulose acetate phthalate, cellulose triacetate, poly (methyl methacrylate), poly (ethyl methacrylate), poly (butyl methacrylate), poly (isobutyl methacrylate), and poly (hexyl methacrylate), poly (isodecyl methacrylate), poly (lauryl methacrylate), poly (phenyl methacrylate), poly (methyl acrylate), poly (isopropyl acrylate), poly (isobutyl acrylate), poly (octadecyl acrylate), beeswax, carnauba wax, paraffin wax, microcrystalline wax, ozokerite; cetostearyl alcohol, stearyl alcohol, cetyl alcohol and myristyl alcohol, glyceryl monostearate; glycerol monooleate, acetylated monoglycerides, tristearin, tripalmitin, cetyl esters wax, glyceryl palmitostearate, glyceryl behenate, zein and hydrogenated vegetable oils.

28. An extended release pharmaceutical composition according to claim 25 wherein the composition additionally contain other pharmaceutically acceptable excipients such as fillers, binders, and lubricants.

29. An extended release tablet suitable for once daily dosing comprising:
a) 50-1500 mg of Linezolid or pharmaceutically acceptable salt, derivative, prodrug, and metabolite thereof;
b) 0.1% to 20% w/w of one or more release controlling materials;
c) one or more other pharmaceutically acceptable excipients.

30. An extended release pharmaceutical composition according to claim 29 wherein the Linezolid is present from about 10% w/w to about 95% w/w of the composition.

31. An extended release pharmaceutical composition according to claim 29 wherein the Linezolid is present from about 50% w/w to about 95% w/w of the composition.

32. An extended release pharmaceutical composition according to claim 29 which, upon oral administration to a human, provides a mean maximum plasma concentration (Cₘₐₓ) of Linezolid not more than about 16 μg/ml.

33. An extended release pharmaceutical composition according to claim 29 which, upon oral administration to a human, provides a mean plasma concentration of at least 2 μg/ml of Linezolid for at least 24 hours.

34. An extended release pharmaceutical composition according to claim 29 wherein release controlling material is selected from group consisting of hydrophilic release controlling materials or hydrophobic release controlling materials or combinations thereof.

35. An extended release pharmaceutical composition according to claim 34 wherein hydrophilic release controlling material is selected from group consisting of hydroxypropyl methylcellulose, hydroxypropyl cellulose, methylcellulose, povidone, polyethylene glycols, vinyl acetate copolymers, polysaccharides as alginates, xanthan gum, chitosan, carrageenan, dextran, polyalkylene oxides as polyethylene oxide, methaacrylic acid copolymers, maleic anhydride/methyl vinyl ether copolymers, carbomers.

36. An extended release pharmaceutical composition according to claim 34 wherein hydrophobic material is selected from group consisting of polyvinyl acetate dispersion, ethyl cellulose, cellulose acetate, cellulose propionate (lower, medium or higher molecular weight), cellulose acetate propionate, cellulose acetate butyrate, cellulose acetate phthalate, cellulose triacetate, poly (methyl methacrylate), poly (ethyl methacrylate), poly (butyl methacrylate), poly (isobutyl methacrylate), and poly (hexyl methacrylate), poly (isodecyl methacrylate), poly (lauryl methacrylate), poly (phenyl methacrylate), poly (methyl acrylate), poly (isopropyl acrylate), poly (isobutyl acrylate), poly (octadecyl acrylate), beeswax, carnauba wax, paraffin wax, microcrystalline wax, ozokerite; cetostearyl alcohol, stearyl alcohol, cetyl alcohol, myristyl alcohol, glyceryl monostearate; glycerol monooleate, acetylated monoglycerides, tristearin, tripalmitin, cetyl esters wax, glyceryl palmitostearate, glyceryl behenate, zein and hydrogenated vegetable oils.

37. An extended release pharmaceutical composition according to claim 34 wherein the composition additionally contain other pharmaceutically acceptable excipients such as fillers, binders, and lubricants.

38. An extended release pharmaceutical composition according to claim 34 wherein the tablet is prepared by direct compression or dry granulation or wet granulation or melt granulation.

39. An extended release pharmaceutical composition suitable for once daily dosing comprising Linezolid or pharmaceutically acceptable salt, derivative, prodrug, metabolite and polymorph thereof and one or more pharmaceutically acceptable excipients so that upon oral administration the maximum concentrations (Cₘₐₓ) of Linezolid in plasma are statistically significantly lower than the immediate release formulation given twice daily, and area under the plasma concentration-time curve (AUC) and the minimum plasma concentration are maintained over 24 hours.

40. An extended release pharmaceutical composition according to claim 39 wherein the Linezolid is present from about 50 mg to about 1700 mg.

41. An extended release pharmaceutical composition according to claim 39 wherein the Linezolid is present from about 300 mg to about 1500 mg.

42. A method of treating bacterial infections in a mammal comprising administering an extended release, pharmaceutical composition suitable for once daily dosing comprising Linezolid or pharmaceutically acceptable salt, derivative, prodrug, metabolite and polymorph thereof as an active ingredient, and one or more pharmaceutically acceptable excipients, capable of maintaining T>MIC for at least 24 hours.

43. An extended release pharmaceutical composition according to claim 42 wherein the Linezolid is present from about 50 mg to about 1700 mg.

44. An extended release pharmaceutical composition according to claim 42 wherein the Linezolid is present from about 300 mg to about 1500 mg.
